(19) United States
(12) Patent Application Publication
(10) Pub. No.: US 2016/0310644 A1
(43) Pub. Date: Oct. 27, 2016

(54) METHODS FOR MAKING DRUG-CONTAINING POROUS SILICONE STRUCTURES

(71) Applicant: ProMed Molded Products, Inc., Plymouth, MN (US)

(72) Inventors: James H. ARPS, Chanhassen, MN (US); Matthew A. PETERSEN, Minneapolis, MN (US)

(21) Appl. No.: 15/135,034

(22) Filed: Apr. 21, 2016

Related U.S. Application Data
(60) Provisional application No. 62/150,710, filed on Apr. 21, 2015.

(51) Int. Cl. A61L 31/16; B29C 45/16; A61L 31/14; A61K 9/00; A61L 31/10

(52) U.S. Cl. CPC A61L 31/16; A61K 9/0036; A61L 31/10; A61L 31/146; B29C 45/1679; A61L 2300/204; A61L 2420/02; A61L 2300/256; B29K 2019/00

(57) ABSTRACT

The invention provides methods for making silicone structures, drug delivery devices and active pharmaceutical agent delivery systems and medical devices containing one or more active pharmaceutical ingredients. The invention provides silicone structures, drug delivery devices and active pharmaceutical agent delivery systems containing one or more active pharmaceutical ingredients. The silicones used in the invention are foaming silicones, which generate void-forming gases which completely volatilize during curing. The silicone structures, drug delivery devices and active pharmaceutical agent delivery systems can be of any shape or size, and are porous, and are capable of being loaded with high doses of the desired drug or drugs. The active pharmaceutical ingredients can be incorporated into the silicone prior to curing, or subsequent to curing, via a soaking process. Additionally, the rate of drug release from the silicone structures, drug delivery devices and active pharmaceutical agent delivery systems of the invention can be controlled by a post-curing membrane or sheath surrounding the structures. Further provided are methods of treating disease in mammals, preventing the onset of disease in mammals and lessening the severity of disease in mammals, by implanting one or more structures, drug delivery devices and active pharmaceutical agent delivery systems into the mammal.

### BACKGROUND OF THE INVENTION

The administration of therapeutic agents, active pharmaceutical agents (APIs) or active agents is a cornerstone of modern medical care. Active agents can serve many purposes including preventing or treating infection, modulating the immune response of the patient, modulating tissue growth, as well as countless other purposes.

Implantable medical devices or structures are now commonly used to deliver active agents to tissues of the body. When delivered from an implantable medical device, active agents can be administered in a site-specific manner because the medical device can be positioned as desired within the body of a patient. Site specific administration can be advantageous because therapeutic effects on target tissues can be enhanced while side effects on other tissues can be decreased. In addition, some medical devices can enable the delivery of an active agent over an extended period of time in order to optimize therapeutic effect.

### SUMMARY OF THE INVENTION

The invention provides methods of making a silicone structures and drug delivery devices containing a pharmaceutical comprising mixing an effective amount of an active pharmaceutical ingredient into a first part of an uncured silicone foam prior to curing, mixing the first part of uncured silicone foam with a second part of uncured silicone foam to make a final mixture, forming a structure from the final mixture, and curing the structure.

Also provided are methods of making a silicone structure and drug delivery devices containing a pharmaceutical, as provided above, and mixing the active pharmaceutical ingredient into a second part of uncured silicone in a separate container from the first part of uncured silicone. In certain embodiments, the silicone structure is a drug delivery device or an active pharmaceutical delivery system.

In certain embodiments, the silicone structures and drug delivery devices are formed by injection molding or extrusion. In other embodiments, the silicone structures are cured at a temperature from about 15° C. to about 300° C. In still other embodiments, the silicone foam is a liquid silicone rubber or a high consistency rubber, or a combination thereof. In certain methods, the mixing of the pharmaceutical ingredient into the silicone is done by blade mixing, dual symmetric planetary mixing and roll mixing.

Some embodiments provide methods where the effective amount of the active pharmaceutical ingredient incorporated into the structure or drug delivery device is about 30% by weight. In addition, some embodiments provide methods where an effective amount of a second active pharmaceutical ingredient is mixed into a first part of uncured silicone foam prior to curing. Still other embodiments provide methods where an effective amount of a second pharmaceutical ingredient is mixed into a second part of uncured silicone foam prior to curing. Further provided is a structure made by any of the methods provides or described herein.

Also provided are methods of making a silicone structure containing a pharmaceutical comprising mixing an effective amount of an active pharmaceutical ingredient into a first part of an uncured silicone foam prior to curing, mixing the first part of uncured silicone foam with a second part of uncured silicone foam to make a final mixture, forming a structure from the final mixture, curing the structure, submerging the structure in a solution comprising a second active pharmaceutical ingredient, squeezing or compressing the structure mechanically, soaking the structure in the solution for an amount of time, removing the structure from the solution, and removing residual solvent from the structure by drying.

Also provides any of the methods provided above or described herein, further comprising submerging in structure in a solution comprising a third active pharmaceutical ingredient, compressing or squeezing the structure mechanically, soaking the structure in the solution comprising a third active pharmaceutical ingredient for an amount of time, removing the structure from the solution, and removing residual solvent from the structure. In certain embodiments, the residual solvent can be removed from the structure by drying. In some methods, drying is freeze drying, air drying or vacuum drying.

According to any of the methods provided or described herein, the solution may optionally comprise a surfactant. In other embodiments of the methods herein, the solution is a water-based solution or a solvent-based solution.

In any of the methods provided or described herein, optionally, the structure or drug delivery device can be placed into a mold, wherein the mold is larger in size than the structure and completely surrounds the structure, the mold is then sealed, a silicone material is injected into the mold, wherein the silicone forms a single layer sheath around the exterior of the structure, and the silicone is cured for an amount of time. Optionally, the sheath the rate at which an active pharmaceutical ingredient is released.

In other embodiments of the methods provided, an active pharmaceutical ingredient is an antiviral agent, an antiretroviral agent, an antimicrobial agent, an antibiotic, an anticancer agent, an anti-inflammatory agent, an antifungal agent, or an anti-angiogenesis agent. In still other embodiments of the methods, an active pharmaceutical ingredient is an antibiotic or an antibody.

Also provided are methods of treating a disease in an animal suffering from said disease, comprising implanting into the animal a structure made by any of the methods provided, described or contemplated herein.

Further provided are methods of preventing the onset of a disease in an animal, comprising implanting into the animal a structure made by any of the methods provided, described or contemplated herein.

In addition, provided are methods of lessening the severity of the symptoms of a disease in an animal suffering from a disease, comprising implanting into the mammal a structure made by any of the methods provided, described or contemplated herein.

In any of the methods provided, described or contemplated herein, the animal is a mammal. In any of the methods provided, described or contemplated herein, the mammal is a human. In any of the methods provided, described or contemplated herein, the structure is a ring.

Also provided is a drug delivery device prepared by any of the methods of processes provided herein. Further provided is an active pharmaceutical agent delivery system prepared by any of the methods of processes provided herein.

In certain embodiments, provides are methods of making an active pharmaceutical agent delivery system comprising forming a porous reservoir body and inserting one or more active pharmaceutical agents within the porous reservoir body. Optionally, these methods comprise a cured silicone sheath around the delivery system.

Additionally, embodiments provided include an active pharmaceutical agent delivery system comprising a reservoir body defining a plurality of pores, the reservoir body comprising one or more silicone foams, and an amount of one or more active pharmaceutical agents disposed within the pores of the reservoir body. Optionally, the drug delivery systems may comprise a cured silicone sheath around said delivery system.

Certain other embodiments include a drug delivery device comprising a device body defining a plurality of pores, the device body comprising one or more silicone foams, the device body have at least one pore opening to an exterior of said device body; and at least one active pharmaceutical ingredient, wherein the at least one active pharmaceutical ingredient is incorporated into the device body. The drug delivery devices described herein may optionally have a cured silicone sheath around the device body.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 illustrates the foam's ability to be molded to shape (here a cylinder) containing numerous external pores and internal cavities that can be used for encapsulation, according to one or more embodiments.

FIG. 2 illustrates the structure of the internal foam cavities. Internal foam cavities are a mixture of interconnected and isolated in structure and vary in size from multiple millimeters to tens of microns in size, according to one or more embodiments.

FIG. 3 is a graph of the relative pore size distribution of silicone foam made, according to one or more embodiments.

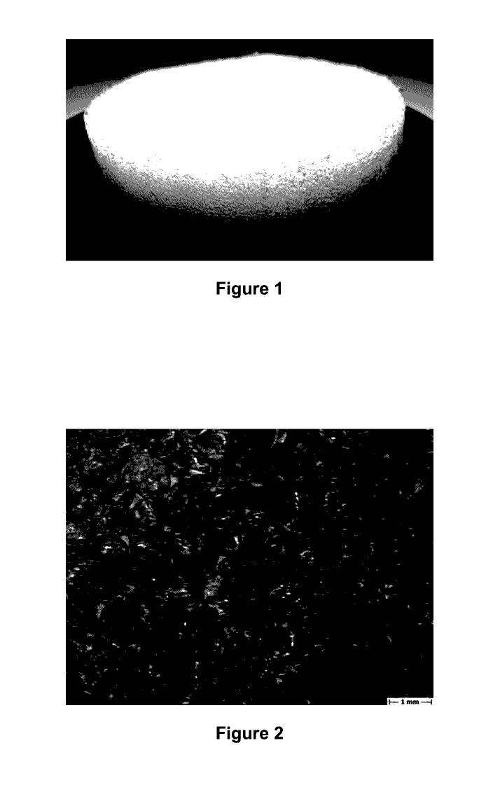

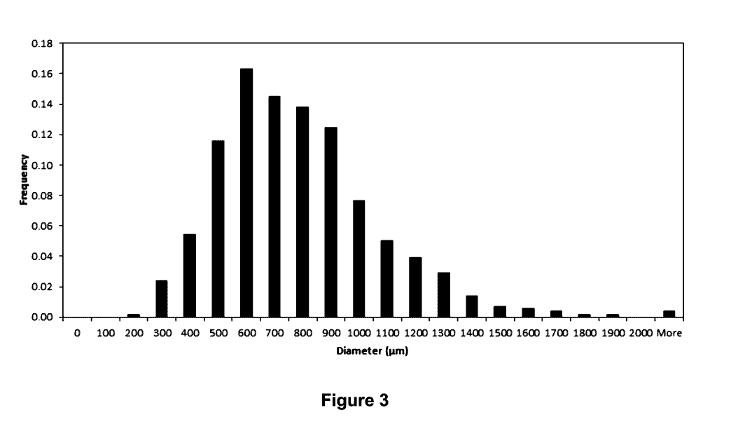

### DETAILED DESCRIPTION OF THE INVENTION

### Definitions

As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage. For example, one or more substituents on a phenyl ring refer to one to five, or one to four, for example if the phenyl ring is disubstituted.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percents, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an amount effective can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” extend to prophylaxis and include prevent, prevention, preventing, lowering, stopping or reversing the progression or severity of the condition or symptoms being treated. As such, the term “treatment” includes medical, therapeutic, and/or prophylactic administration, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

The compound can be conveniently administered in a unit dosage form, for example, containing 5 to 1000 mg/m², conveniently 10 to 750 mg/m², most conveniently, 50 to 500 mg/m² of active ingredient per unit dosage form. When included during compounding, API can be loaded in the range from 0-50% by weight. When included by post-soaking, API can be included from the range of 0-30% by weight. Loading by post-soaking may include soaking of API dispersed in either organic or aqueous solvent followed by removal of solvent by passive evaporation, reduced pressure, increased temperature, or lyophilization, or a combination thereof. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As used herein, “active pharmaceutical ingredient” and API, are used interchangeably, and refer to a substance in a pharmaceutical compound that is biologically active. Some pharmaceutical compounds may contain more than one active pharmaceutical ingredient. An active pharmaceutical ingredient may also be defined as any substance or mixture of substances intended to be used in the manufacture of a drug product and that, when used in the production of a drug, becomes an active ingredient in the drug product. Such substances are intended to furnish pharmacological activity or other direct effect in the diagnosis, cure, mitigation, treatment or prevention of disease or to affect the structure and function of the body. Other terms may be used to refer to active pharmaceutical ingredient, such as drug substance.

Silicone has become well-established as an implantable device material for controlled delivery of a wide range of drugs due to its biocompatibility, chemical inertness, capacity for controlled release, and amenability to formation into a range of geometries by such techniques as molding and extrusion. By appropriate selection of parameters such as material composition, drug formulation, device geometry, drug load, and/or incorporation of a rate-controlling membrane, silicone devices can be fashioned in such a way as to gradually and predictably release drugs over time periods ranging from days to years.

As used herein, “silicone” refers to polymers that include any inert, synthetic compound made up of repeating units of siloxane, which is a molecule of two silicon atoms and one oxygen atom frequently combined with carbon and/or hydrogen. More precisely called polymerized siloxanes or polysiloxanes, silicones are mixed inorganic-organic polymers with the chemical formula [R₂SiO]ₙ, where R is an organic group such as methyl, ethyl, or phenyl. These materials consist of an inorganic silicon-oxygen backbone chain ( . . . —Si—O—Si—O—Si—O— . . . ) with organic side groups attached to the silicon atoms, which are four-coordinate. In some cases, organic side groups can be used to link two or more of these —Si—O— backbones together. By varying the —Si—O— chain lengths, side groups, and crosslinking, silicones can be synthesized with a wide variety of properties and compositions. They can vary in consistency from liquid to gel to rubber to hard plastic. The silicones used in the embodiments of the invention are pharmaceutically acceptable and are biocompatible. Silicone curing may be facilitated by platinum- or tin-based catalysts. Such catalysts are readily available for commercial purchase, and are art recognized for this purpose. In the silicones contemplated for use in the products and methods of the invention, foaming gas may evolve as part of the chemistry used for silicone curing (including, as a non-limiting example, BlueStar Silbione RT Foams or similar foam chemistry) or foaming may occur as a result of including an additive (including, as a non-limiting example, a foam such as Wacker SILFOAM or a similar product). Foaming gas may be comprised of hydrogen, carbon dioxide, water, or a combination thereof, or other volatile species. Molding of the silicone may be performed using metal (as a non-limiting example, metals such as steel or aluminum) tooling that dictate the shape of the final part, or in polymeric containers (especially polypropylene, polyethylene, or polyacetal), or in shapes from which the final part is cut out, or extruded to an essentially cylindrical shape, or a combination thereof.

Generally, a drug is incorporated into a continuous polymeric matrix by compounding with uncured silicone prior to inducing cure. After completion of this reaction, the silicone generally forms a solid polymeric matrix enclosing a combination of dispersed particles and molecularly dissolved drug. Release then occurs by diffusion through the polymer matrix to the periphery of the part, where it can interact with the surrounding tissue to achieve its desired therapeutic effect.

Existing approaches are limited in several key regards. In applications requiring delivery of a poorly bioavailable or sparingly soluble drug or API, the low surface area presented by a conventional continuous polymer matrix can limit dissolution and negatively impact therapeutic efficacy. At high drug or API loadings or in the presence of drugs or APIs containing interfering chemistries, crosslinking of the polymer matrix can be disrupted, leading to a loss of mechanical integrity. Finally, the elevated temperatures, curing chemistries, and processing conditions required to induce crosslinking can have a deleterious effect on sensitive drugs or APIs if they are incorporated prior to curing. These issues are further exacerbated when the delivery of large molecule therapeutic agents such as proteins, antibodies, and nucleic acids are desired. Large molecules (such as those molecules greater than 1 kDa in molecular weight) incorporated into a dense silicone matrix will release slowly if at all due to their size limited mobility due to crosslinking reactions. Further, for biological payloads, even modest curing temperatures greatly increase the likelihood of denaturing or degrading the drug or API, functional groups may hinder the crosslinking reaction, and low solubilities would be expected for many such molecules in the nonpolar silicone matrix.

Applicants have unexpectedly discovered several means of incorporating drugs or APIs into porous, very high surface area silicone foams. By utilizing chemistries capable of generating void-forming gases or additives which completely volatilize during the curing process, it is possible to create porous rings that can be loaded with very high drug or API doses while not substantially compromising mechanical integrity. These rings, as well as other structures made using Applicants' technologies, are also referred to herein as drug delivery devices or active pharmaceutical agent delivery systems. For purposes of this writing, “drug delivery device,” “active pharmaceutical agent delivery system,” “structure,” and “silicone structure” can be used interchangeably. By use of appropriate processing conditions it is possible to control the number, size, and shape of these voids as well as their interconnectedness, allowing control of overall density, drug loading, and release by diffusion through both pores and the polymer matrix. Further control over drug or API release is possible by forming a rate-controlling membrane over the part, either via a thin layer formed at the interface with a mold during initial curing or by over molding of a rate-controlling layer subsequent to drug or API loading. This novel approach allows incorporation into the structures of much higher loads of previously problematic drugs, including large molecule APIs, control over their release, and potential enhancements to drug bioavailability while using equipment and procedures widely used for silicone molding and processing.

APIs or drugs can be incorporated into one of two points in the silicone foam molding or extrusion process, though sequential use of both for incorporation of multiple payloads is also possible. First, the API or drug can be mixed into the uncured silicone prior to curing, typically by compounding with one or both of the two parts (A & B) in which silicone is generally supplied which react only when mixed together. Optionally, the API or drug can be mixed into the uncured silicone prior to curing, typically by compounding with three parts (A, B, & C) in which silicone is generally supplied which react only when mixed together. Silicones compatible with manufacturing as foams include both liquid silicone rubber (LSR) and high consistency rubber (HCR). Examples of silicone foams that can be used as a basis for drug delivery include Silbione RT 4241 (Bluestar Silicones), ELASTOSIL® AUX foaming batches MTB, XTB, and BTB (Wacker Silicones), and MED-2310P (Nusil Technology). Persons skilled in the art utilize a variety of API or drug compounding techniques for LSRs and HCRs including but not limited to blade mixing, dual asymmetric planetary mixing, and roll milling. Once the API or drug is mixed into the uncured silicone it may be injection molded or extruded at temperatures ranging from ambient to approximately 300° F., being careful to limit any risk of thermal degradation of the API or drug. The resulting cellular foam structure allows greater fluid ingress, API or drug dissolution, and diffusion than a typical dense silicone structure. The estimated maximum drug load or API load which can be incorporated by this when adding API to uncured silicone is approximately 30% by weight, after which mechanical integrity of the molded structure becomes problematic. When incorporating one or more APIs via soaking the structure in a solution of one or more APIs, the maximum drug load or API load will vary, depending on the amount of volume that can be taken up by the structure, as well as the solubility of the one or more APIs desired. The structures can be loaded with any size molecule up to a dimension of approximately 800 microns. In addition, the structures can be loaded with molecules of any diameter, with no minimum size up to a maximum of 800 microns.

### Representative Silicone Foams

A second method is to incorporate the API or drug into preformed silicone foam structures via soaking in an appropriate solution of the API or drug. In the case of biologics, the solutions are generally water-based solutions, which, once absorbed by the foam, are allowed to dry using various methods including freeze-, air-, and vacuum-drying. Further, surfactants may be used to enhance wetting of the hydrophobic silicone surface. Other APIs or drugs may be dissolved in volatile organic solvents which may also have the added benefit of swelling the silicone foam, allowing further uptake of the drug solution. Once fully absorbed, the solvent is allowed to volatilize, leaving the API or drug trapped within the pores of the structure. Drug or API loadings of 50% by weight or more are possible using a soaking method depending on the solubility of the API or drug in the soak solution.

The structures, drug delivery devices and active pharmaceutical agent delivery systems as provided by the representative embodiments of the invention may be utilized for eluting one or more APIs in an animal, such as a human. In certain embodiments, the structures may be active agent or API delivery systems. In other embodiments, the structures may be medical devices or orthopedic devices. As a non-limiting example, the structures may incorporate one or more antibiotics and be implanted at or near an orthopedic surgical site in an animal, and provide for a designed release of the API at the site. As another non-limiting example, the structures may incorporate one or more APIs for use in treating cancer and/or cancer side effects. Thus, in certain embodiments, a structure of the invention may have an anticancer compound and an antifungal compound incorporated into the foam, and is used in an animal for treating cancer and deterring the growth of fungi in the animal.

The silicones and silicone foams used in the invention provided herein may be designed to take the form or structure most appropriate, convenient, effective or desired in order to accomplish the intended result. For example, the silicone foams made are made into the following shapes: rings, spheres, wedges, planks, panels, discs, tablets, rods, needles, or other structures or drug delivery devices. This list is meant for illustrative purposes only; it does not provide a complete list of all of the shapes that made be formed to create the technologies disclosed in this invention. Non-limiting examples include rings that may have outer diameters ranging from at least approximately 58 mm (for human-sized implementations) to approximately 15 mm (for small-animal-sized implementations) with cross sectional diameters of between 2-10 mm.

### Diseases and Treatments

The invention provides therapeutic methods of treating disease in a mammal, which involve administering to a mammal having a disease an effective amount of a compound or composition described herein using a structure, drug delivery device or active pharmaceutical agent delivery system described herein. A mammal includes a primate, human, rodent, canine, feline, bovine, ovine, equine, swine, caprine, bovine and the like. As used herein, “disease” refers to a particular abnormal, pathological condition that affects part or all of an organism. It is often defined as a medical condition associated with specific symptoms and signs. It may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the person afflicted, or similar problems for those in contact with the person. Disease may also refers to injuries, disabilities, disorders, syndromes, infections, isolated symptoms, deviant behaviors, and atypical variations of structure and function. Disease refers to any disease, including but not limited to cancer, tumors, infectious diseases, diseases and conditions caused by bacteria, viruses, or fungi, autoimmune conditions and diseases, syndromes of unknown etiology, deficiency conditions, hereditary conditions, and physiologic conditions.

The term cancer refers to any various type of malignant neoplasm, for example, colon cancer, breast cancer, melanoma and leukemia, and in general is characterized by an undesirable cellular proliferation, e.g., unregulated growth, lack of differentiation, local tissue invasion, and metastasis.

The ability of a compound of the invention to treat a disease or condition may be determined by using assays well known to the art. For example, if the disease is cancer, the design of treatment protocols, toxicity evaluation, data analysis, quantification of tumor cell kill, and the biological significance of the use of transplantable tumor screens are known. In addition, the ability of a device or a therapy to treat cancer may be determined using the Tests as described below.

### Active Pharmaceutical Ingredients

Embodiments of medical devices, structures and delivery systems described herein can elute or release one or more APIs or active agents. As used herein, the term “active agent” or API can also mean a compound that has a particular desired activity. For example, an active agent can be a therapeutic compound that exerts a specific activity on a subject. In some embodiments, active agent will, in turn, refer to a peptide, protein, carbohydrate, nucleic acid, lipid, polysaccharide or combinations thereof, or synthetic inorganic or organic molecule that causes a desired biological effect when administered in vivo to an animal including but not limited to birds and mammals, including humans. Desired biological effects can include, but are not limited to, preventing or treating infection, modulating the immune response of the patient, modulating tissue growth, and the like. Active agent can include macromolecules, small molecules, hydrophilic molecules, hydrophobic molecules, and the like.

Active Pharmaceutical Ingredients, or APIs, or “active agents” refer to any an antiviral agent, an antiretroviral agent, an antimicrobial agent, an anticancer agent, an anti-inflammatory agent, an antifungal agent, or an anti-angiogenesis agent. Further, Active Pharmaceutical Ingredients, or APIs, also refer to antibodies (monoclonal or other), growth factors, cytokines, interferons, interleukins, TNF, DNA or RNA inhibitors, nucleic acids, biotherapeutics, biopharmaceuticals, engineered macromolecular products, proteins, fusion proteins, therapeutic enzymes, vaccines, blood or blood components, allergenics, somatic cells, hormones, gene therapies, tissues, recombinant therapeutic protein and living cells. Active Pharmaceutical Ingredients, or APIs, also refer to nutraceuticals, essential oils, alternative medicines, herbal supplements, and the like. In addition, a combination of any of the above listed agents may be used in the invention provided herein.

According to embodiments of the invention, the medical devices, structures and delivery systems provided herein may be loaded with one or more APIs at a maximum concentration of 30-60% by weight. In patients, the APIs will be released over hours, days, or months, depending on the application, condition treated, and the APIs themselves. The release of the APIs may be of first or zero order, or a combination thereof. In other embodiments, the APIs can release in the form of a “burst” or a high rate of release, followed by a steady elution at desired or specified rate appropriate for the application. In addition, in other embodiments, the release of the APIs may be of one order for one of a plurality of APIs, and another order for another API in the same system, device or structure.

Active agents useful according to the invention include substances that possess desirable therapeutic characteristics for application to the implantation or treatment site. Active agents useful in the present invention can include many types of therapeutics including thrombin inhibitors, antithrombogenic agents, thrombolytic agents, fibrinolytic agents, anticoagulants, anti-platelet agents, vasospasm inhibitors, calcium channel blockers, steroids, vasodilators, anti-hypertensive agents, β-blockers, anti-angina agents, cardiac inotropic agents, anti-arrhythmic agents, lipid regulating agents, antimicrobial agents, antibiotics, antibacterial agents, antiparasitic and/or antiprotozoal agents, antiseptics, antifungals, antimalarials, angiogenic agents, anti-angiogenic agents, inhibitors of surface glycoprotein receptors, antimitotics, microtubule inhibitors, antisecretory agents, actin inhibitors, remodeling inhibitors, antisense nucleotides, anti-metabolites, miotic agents, anti-proliferatives, anticancer chemotherapeutic agents, anti-neoplastic agents, antipolymerases, antivirals, anti-inflammatory steroids or non-steroidal anti-inflammatory agents, analgesics, antipyretics, immunosuppressive agents, immunomodulators, growth hormone antagonists, growth factors, radiotherapeutic agents, peptides, proteins, enzymes, hormones, extracellular matrix components, ACE inhibitors, free radical scavengers, chelators, anti-oxidants, photodynamic therapy agents, gene therapy agents, anesthetics, opioids, dopamine agonists, antihistamines, tranquilizers, anticonvulsants, muscle relaxants, antispasmodics and muscle contractants, anticholinergics, ophthalmic agents, antiglaucoma solutes, prostaglandins, neurotransmitters, imaging agents, specific targeting agents, and cell response modifiers.

Active agents can specifically include anti-microbial agents such as antibiotics. Antibiotics are substances which inhibit the growth of or kill microorganisms. Antibiotics can be produced synthetically or by microorganisms. Examples of antibiotics include penicillin, tetracycline, tobramycin, chloramphenicol, minocycline, doxycycline, vancomycin, bacitracin, kanamycin, neomycin, polymyxin B, gentamycin, erythromycin, geldanamycin, geldanamycin analogs, cephalosporins, or the like. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone. Anti-microbial agents can specifically include anti-microbial peptides. Anti-microbial peptides can include those described in U.S. Pat. Nos. 5,945,507, 6,835,713, and 6,887,847, the contents of which are herein incorporated by reference.

Anti-microbial agents can also include antiseptics. Antiseptics are recognized as substances that prevent or arrest the growth or action of microorganisms, generally in a nonspecific fashion, e.g., either by inhibiting their activity or destroying them. Examples of antiseptics include silver sulfadiazine, chlorhexidine, glutaraldehyde, peracetic acid, sodium hypochlorite, triclosan, phenols, phenolic compounds, iodophor compounds, quaternary ammonium compounds, and chlorine compounds.

Active agents can specifically include antiviral agents. Antiviral agents are substances capable of destroying or suppressing the replication of viruses. Examples of anti-viral agents include α-methyl-1-adamantanemethylamine, hydroxy-ethoxymethylguanine, adamantanamine, 5-iodo-2′-deoxyuridine, trifluorothymidine, interferon, and adenine arabinoside. Active agents can specifically include those agents capable of modulating bone and cartilage tissue growth. By way of example, active agents can include osteogenic growth peptide, insulin-like growth factor-1 (IGF-1), insulin, human growth hormone, activated vitamin D binding protein (ADBP), bone and cartilage stimulating peptide (such as BCSP-1), bone morphogenic proteins (including BMP-7), and platelet derived growth factor (PDGF). Other active agents capable of modulating bone and cartilage can specifically include peptides described in U.S. Pat. No. 5,635,482 (the contents of which is herein incorporated by reference) and commercially available under the trade name P-15.

Active agents can specifically include enzyme inhibitors. Enzyme inhibitors are substances that inhibit an enzymatic reaction. Examples of enzyme inhibitors include edrophonium chloride, N-methylphysostigmine, neostigmine bromide, physostigmine sulfate, tacrine HCL, tacrine, 1-hydroxy maleate, iodotubercidin, p-bromotetramisole, 10-(α-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylaminie, N-monomethyl-L-arginine acetate, carbidopa, 3-hydroxybenzylhydrazine HCl, hydralazine HCl, clorgyline HCl, deprenyl HCl(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-di-phenylvalerate hydrochloride, 3-isobutyl-1-methylxanthne, papaverine HCl, indomethacind, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate R(+), p-aminoglutethlimide tartrate S(−), 3-iodotyrosine, alpha-methyltyrosine L(−), alpha-methyltyrosine D(−), cetazolamide, dichlorphenamide, 6-hydroxy-2-benzothiazolesulfonamide, and allopurinol.

Active agents can specifically include anti-pyretics. Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide.

Active agents can specifically include anesthetics. Local anesthetics are substances that have an anesthetic effect in a localized region. Examples of such anesthetics include procaine, lidocaine, tetracaine and dibucaine.

Active agents can specifically include imaging agents. Imaging agents are agents capable of imaging a desired site, e.g., tumor, in vivo. Examples of imaging agents include substances having a label that is detectable in vivo, e.g., antibodies attached to fluorescent labels. The term antibody includes whole antibodies or fragments thereof.

Active agents can specifically include cell response modifiers. Cell response modifiers include chemotactic factors such as platelet-derived growth factor (PDGF). Other cell response modifiers can include neutrophil-activating protein, monocyte chemoattractant protein, macrophage-inflammatory protein, SIS (small inducible secreted), platelet factor, platelet basic protein, melanoma growth stimulating activity, epidermal growth factor, transforming growth factor alpha, fibroblast growth factor, platelet-derived endothelial cell growth factor, insulin-like growth factor, nerve growth factor, bone growth/cartilage-inducing factor (alpha and beta), and matrix metalloproteinase inhibitors. Other cell response modifiers include the interleukins, interleukin receptors, interleukin inhibitors, interferons, including alpha, beta, and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins, antisense molecules, androgenic receptor blockers and statin agents.

Other active agents can include heparin, covalent heparin, synthetic heparin salts, or another thrombin inhibitor; hirudin, hirulog, argatroban, D-phenylalanyl-L-poly-L-arginyl chloromethyl ketone, or another antithrombogenic agent; urokinase, streptokinase, a tissue plasminogen activator, or another thrombolytic agent; a fibrinolytic agent; a vasospasm inhibitor; a calcium channel blocker, a nitrate, nitric oxide, a nitric oxide promoter, nitric oxide donors, dipyridamole, or another vasodilator; HYTRIN® or other anti hypertensive agents; a glycoprotein IIb/IIIa inhibitor (abciximab) or another inhibitor of surface glycoprotein receptors; aspirin, ticlopidine, clopidogrel or another antiplatelet agent; colchicine or another antimitotic, or another microtubule inhibitor; dimethyl sulfoxide (DMSO), a retinoid, or another antisecretory agent; cytochalasin or another actin inhibitor; cell cycle inhibitors; remodeling inhibitors; deoxyribonucleic acid, an antisense nucleotide, or another agent for molecular genetic intervention; methotrexate, or another antimetabolite or antiproliferative agent; tamoxifen citrate, TAXOL®, paclitaxel, or the derivatives thereof, rapamycin (or other rapalogs e.g. ABT-578 or sirolimus), vinblastine, vincristine, vinorelbine, etoposide, tenopiside, dactinomycin (actinomycin D), daunorubicin, doxorubicin, idarubicin, anthracyclines, mitoxantrone, bleomycin, plicamycin (mithramycin), mitomycin, mechlorethamine, cyclophosphamide and its analogs, chlorambucil, ethylenimines, methylmelamines, alkyl sulfonates (e.g., busulfan), nitrosoureas (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, morpholino phosphorodiamidate oligomer or other anti-cancer chemotherapeutic agents; cyclosporin, tacrolimus (FK-506), pimecrolimus, azathioprine, mycophenolate mofetil, mTOR inhibitors, or another immunosuppressive agent; cortisol, cortisone, dexamethasone, dexamethasone sodium phosphate, dexamethasone acetate, dexamethasone derivatives, betamethasone, fludrocortisone, prednisone, prednisolone, 6U-methylprednisolone, triamcinolone (e.g., triamcinolone acetonide), or another steroidal agent; trapidil (a PDGF antagonist), angiopeptin (a growth hormone antagonist), angiogenin, a growth factor (such as vascular endothelial growth factor (VEGF)), or an anti-growth factor antibody (e.g., ranibizumab, which is sold under the tradename LUCENTIS®), or another growth factor antagonist or agonist; dopamine, bromocriptine mesylate, pergolide mesylate, or another dopamine agonist; iodine-containing compounds, barium-containing compounds, gold, tantalum, platinum, tungsten or another heavy metal functioning as a radiopaque agent; a peptide, a protein, an extracellular matrix component, a cellular component or another biologic agent; captopril, enalapril or another angiotensin converting enzyme (ACE) inhibitor; angiotensin receptor blockers; enzyme inhibitors (including growth factor signal transduction kinase inhibitors); ascorbic acid, alpha tocopherol, superoxide dismutase, deferoxamine, a 21-aminosteroid (lasaroid) or another free radical scavenger, iron chelator or antioxidant; a ¹⁴C—, ³H—, ¹³¹I—, ³²P— or ³⁶S-radiolabelled form or other radiolabelled form of any of the foregoing; an estrogen (such as estradiol, estriol, estrone, and the like) or another sex hormone; AZT or other antipolymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluorozinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin, or other antibody targeted therapy agents; gene therapy agents; enalapril and other prodrugs; PROSCARR®, HYTRINR® or other agents for treating benign prostatic hyperplasia (BHP); mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of or intermediates of any of these. Active agents can specifically include microparticles. For example, active agents, such as those described above, can be formulated as microparticles and disposed within interconnected pores.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

The silicone components used in the inventions and embodiments provided are readily available commercial products, and can be in any form (powder, gel, liquid, and other forms) appropriate for the application. As one non-limiting example, Silbione® RT (Bluestar Silicones, U.S.A.) can be used as the silicone in the medical devices, structures and delivery systems provided herein.

### Example 1

### Manufacture of a Dapivirine-Maraviroc Vaginal Ring by Compounding Method

Using silicone containing an appropriate foaming agent, approximately 9.3 g of silicone ‘A’ component, 70 mg of dapivirine, and 630 mg maraviroc were weighed into a 20 mL cylindrical container and roughly mixed by hand. In a separate 20 mL cylindrical container, approximately 9.3 g of silicone ‘B’ component, 70 mg of dapivirine, and 630 mg maraviroc were also weighed and roughly mixed by hand. Each container was in turn subjected to treatment in a dual asymmetric mixer (Flaktek SpeedMixer) for 1-4 minutes at up to 3500 RPM to disperse drug. Each mix was then transferred to a 10 mL dispenser. The ‘A’ and ‘B’ dispensers were then attached to a static mixing apparatus. A total of 0.5-5 g of combined mix was subsequently injected through the static mixer and evenly dispersed into an open mold describing a ring shape. The mold was then closed, clamped, and held between 20 and 70° C. Material was subsequently allowed to cure for 40-80 minutes before demolding. A wide range of curing times is possible for the embodiments of this invention. As a non-limiting example, some in embodiments, the material can be allowed to cure for approximately 2-3 minutes, or 2-5 minutes, or 5-10 minutes, or 10-15 minutes, or 15-20 minutes, or 20-25 minutes, 25-30 minutes, 30-35 minutes, 35-40 minutes, 40-45 minutes, 45-50 minutes, 50-55 minutes, 55-60 minutes, 60-65 minutes, 65-70 minutes, 70-75 minutes, or 75-80 minutes. The process was then repeated until all of the mix was depleted.

### Example 2

### Manufacture of a Dapivirine-Maraviroc Vaginal Ring by Postsoaking Method

Using silicone containing an appropriate foaming agent, approximately 9.9 g of silicone ‘A’ component and 70 mg of dapivirine were weighed into a 20 mL cylindrical container and roughly mixed by hand. In a separate 20 mL cylindrical container, approximately 9.9 g of silicone ‘B’ component and 70 mg of dapivirine were also weighed and roughly mixed by hand. The contents of each container were treated in a dual asymmetric mixer (Flaktek SpeedMixer) for 1-4 minutes at up to 3500 RPM to disperse drug. Each mix was then transferred to a 10 mL dispenser. The ‘A’ and ‘B’ dispensers were then attached to a static mixing apparatus. A total of 0.5-5 g of combined mix was subsequently injected through the static mixer and evenly dispersed into an open mold describing a ring shape. The mold was then closed, clamped, and held between 20 and 70° C. Material was subsequently allowed to cure for 40-80 minutes before demolding. The process was then repeated until all mix was depleted.

Maraviroc was dispersed into 0-100% isopropanol in heptane at a concentration of 5-20 mg/mL solution and a total volume of at least 10 mL. Rings were submerged in the solution and mechanically squeezed to expel gas trapped in the void volume and take up the isopropanol-heptane solution. Rings were subsequently allowed to soak up solution for 5-60 minutes. Rings were then removed from solution and residual solvent removed either via passive evaporation or under 5-30 in Hg vacuum.

### Example 3

### Manufacture of an Antibody-Delivering Vaginal Ring

Using a ring formed using either of the above methods, a solution was prepared containing an anti-HIV antibody (mouse monoclonal anti-HIV gp160) at concentrations from 0.5 to 200 μg/mL, 0-0.02 w/v % of the nonionic surfactant Polysorbate 20, and other preservatives and stabilizers commonly used in antibody dispersion by those skilled in the art. Rings were submerged in the solution and mechanically squeezed to expel gas trapped in the void volume and take up the aqueous solution. Rings were then removed from solution and solvent removed either by passive evaporation, under 5-30 in Hg vacuum, or lyophilized.

### Example 4

### Overmolding of a Rate-Controlling Membrane

Rings created using any of the above methods were inserted into an appropriately-sized mold describing the shape of the ring plus 0.1-3 mm of additional space surrounding the cross-section of the ring. The mold was then sealed, silicone material injected, and material cured at 20-200° C. for 30-90 s to form a sheath surrounding the central foam portion. In certain embodiments, the mold was sealed, silicone material injected, and material cured at 20-200° C. for approximately 60 seconds to form a sheath surrounding the central foam portion. One non-limiting example of accomplishing the overmolding is via a single injection to form the entire membrane or via a two-part process consisting of first forming one half of the sheath, placing the foam ring inside of this half-toroid, and subsequently overmolding these two pieces to simultaneously form the other half of the membrane and bond the whole into a single piece.

In certain embodiments, the sheath is molded over an antibody or protein-socked ring. Rings where the sheath is molded after the antibody or protein has been allowed to soak in the presence of the ring are especially useful for peptides or other biologics where secondary structure is not a major concern.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

We claim:

1. A drug delivery device comprising:
(a) a device body defining a plurality of pores, the device body comprising one or more silicone foams and having at least one pore opening to an exterior of said device body; and
(b) an effective amount of at least one active pharmaceutical agent, wherein the at least one active pharmaceutical agent is incorporated into the device body; and
optionally comprising a cured silicone sheath surrounding the exterior of the device body.

2. The drug delivery device of claim 1, wherein the at least one active pharmaceutical agent is an antibody, an antiviral agent, an antiretroviral agent, an antimicrobial agent, an antibiotic, an anticancer agent, an anti-inflammatory agent, an antifungal agent, or an anti-angiogenesis agents, or a combination thereof.

3. A method of making the drug delivery device of claim 1, comprising:
(a) forming a device body from one or more silicone foams, the device body having a plurality of pores and at least one pore opening to an exterior of said device body; and
(b) incorporating an effective amount of at least one active pharmaceutical ingredient into the pores of said device body, and, optionally,
(c) forming a sheath of cured silicone around the exterior of the device body.

4. An active pharmaceutical agent delivery system comprising:
(a) a reservoir body defining a plurality of pores, the reservoir body comprising one or more silicone foams and having at least one pore opening to an exterior of said body; and
(b) an effective amount of one or more active pharmaceutical agents disposed within the pores of the reservoir body; and
optionally comprising a cured silicone sheath around the exterior of the reservoir body.

5. The drug delivery system of claim 1, wherein the at least one active pharmaceutical agent is an antibody, an antiviral agent, an antiretroviral agent, an antimicrobial agent, an antibiotic, an anticancer agent, an anti-inflammatory agent, an antifungal agent, or an anti-angiogenesis agent.

6. A method of making the active pharmaceutical agent delivery system of claim 4, comprising:
(a) forming a porous reservoir body from one or more silicone foams; and
(b) inserting an effective amount of one or more active pharmaceutical agents within the pores of the porous reservoir body, and optionally,
(c) forming a sheath of cured silicone around the exterior of the device body.

7. A method of making a silicone structure containing an effective amount of at least one active pharmaceutical agent, comprising:
(a) contacting at least one active pharmaceutical agent and a first uncured silicone foam component, sufficient to form a first mixture;
(b) mixing a second part of uncured silicone foam with the first mixture to make a final mixture;
(c) forming a structure from the final mixture; and
(d) curing the structure.

8. The method of claim 7, wherein the second part of uncured silicone foam is mixed with an effective amount of at least one active pharmaceutical agent prior to mixing with the first mixture.

9. The method of claim 7, wherein the structure is formed by injection molding or extrusion.

10. The method of claim 7, wherein the structure is cured at a temperature from about 15° C. to about 300° C.

11. The method of claim 7, wherein the silicone foam is a liquid silicone rubber or a high consistency rubber, or a combination thereof.

12. The method of claim 7, wherein the mixing is done by blade mixing, dual symmetric planetary mixing, roll mixing or a combination thereof.

13. The method of claim 7, wherein the effective amount of the one or more active pharmaceutical agents incorporated into the structure is about 30% by weight.

14. The method of claim 7, wherein an effective amount of a second active pharmaceutical agent is mixed with the first part of uncured silicone at (a), or the second part of uncured silicone at (b) prior to mixing with the first mixture.

15. The method of claim 7, further comprising:
(e) submerging the structure in a solution comprising an effective amount of a second active pharmaceutical agent;
(f) squeezing the structure mechanically;
(g) soaking the structure in the solution for an amount of time;
(h) removing the structure from the solution;
(i) removing residual solvent from the structure by freeze drying, air drying or vacuum drying; and optionally, submerging the structure in a solution comprising an effective amount of a third active pharmaceutical agent and repeating steps (f) through (i).

16. The method claim 7, further comprising:
(e) placing the structure into a mold, wherein the mold is larger in size than the structure and completely surrounds the structure;
(f) sealing the mold;
(g) injecting a silicone material into the mold, wherein the silicone forms a single layer sheath around the exterior of the structure;
(h) curing the silicone for an amount of time.

17. The sheath of claim 16, wherein the sheath controls the rate at which an active pharmaceutical ingredient is released.

18. The method of claim 16, wherein the solution comprises a surfactant and the solution is water-based or solvent-based.
